(19) United States
(12) Patent Application Publication
(10) Pub. No.: US 2015/0273453 A1
(43) Pub. Date: Oct. 1, 2015

(54) BISMUTH-MODIFIED MOLECULAR SIEVES AND METHODS FOR PREPARING AND USING BISMUTH-MODIFIED MOLECULAR SIEVES

(71) Applicant: UOP LLC, Des Plaines, IL (US)

(72) Inventor: Hui Wang, Buffalo Grove, IL (US)

(73) Assignee: UOP LLC, Des Plaines, IL (US)

(21) Appl. No.: 14/225,363

(22) Filed: Mar. 25, 2014

(51) Int. Cl. B01J 29/78; C07C 2/66

(52) U.S. Cl. CPC B01J 29/78; C07C 2/66

(57) ABSTRACT

Bismuth-modified molecular sieve catalysts and methods for preparing and using bismuth-modified molecular sieve catalysts are provided. In one embodiment, a bismuth-modified molecular sieve catalyst includes a molecular sieve having an external surface. The molecular sieve is selected from the group consisting of MFI, MEL, MOR, MTW, BEA, CHA, FAU, EMT, MTT, MWW, TON, TUN, EUO, IMF and FER framework types. The bismuth-modified molecular sieve catalyst further includes a coating on the external surface. The coating comprises a bismuth-containing material.

### TECHNICAL FIELD

The technical field generally relates to surface-modified molecular sieves and their preparation and use, and more particularly relates to molecular sieves having external surfaces modified with bismuth, and to methods for the preparation and use of such bismuth-modified molecular sieves.

### BACKGROUND

Molecular sieves include zeotypes, such as alumino-silicate zeolites, alumino-phosphate (ALPO) molecular sieves, silico-alumino-phosphate (SAPO) molecular sieves, and metallo-alumino-phosphate (MeAPO) molecular sieves, that are porous oxide structures having well-defined pore structures due to a high degree of crystallinity. Conventional molecular sieves may be naturally formed or synthesized. Exemplary crystalline alumino-silicate zeolites include those having alumino-silicate cage structures in which alumina and silica tetrahedra are intimately connected with each other in an open three-dimensional crystalline network. The tetrahedra are cross-linked by the sharing of oxygen atoms, with spaces between the tetrahedra occupied by water molecules prior to partial or total dehydration of the zeolite. Dehydration results in crystals interlaced with channels having molecular dimensions. In a hydrated form, the crystalline alumino-silicate zeolites are generally represented by the formula, M_{2/n}O:Al₂O₃:wSiO₂:yH₂O, where “M” is a cation that balances the electrovalence of the tetrahedra and is generally referred to as an exchangeable cationic site, “n” represents the valence of the cation, “w” represents the moles of SiO₂, and “y” represents the moles of water. The exact structure type of an alumino-silicate zeolite is generally identified by the particular silica:alumina molar ratio (SiO₂/Al₂O₃) and the pore dimensions of the cage structures. Cations occupying exchangeable cationic sites in the zeolite may be replaced with other cations by ion exchange methods well known to those having ordinary skill in the field of crystalline alumino-silicates.

Molecular sieves may be formed as crystalline particles by mixing a fine powder form of the molecular sieve with a binder. The binder may be an amorphous inorganic material, such as silica, alumina, titania, zirconia, alumino-phosphate or certain clays and mixtures thereof “Formed molecular sieves” may be extrudates, tablets, oil drops, microspheres, spheres, beads, or the like. The molecular sieves may be formed by oil-dropping, spray-drying, extrusion, or other “forming” techniques.

Molecular sieves are commonly used as an active component in various catalytic applications. Molecular sieves may also be used for adsorption and separation processes. It has been recognized that the surfaces of certain molecular sieves can be modified to selectively reduce surface activity as well as to enhance molecular-sieving or shape-selective capability. Generally, surface-modified molecular sieves can more efficiently catalyze selected reactions than non-modified molecular sieves. Molecular sieve surface modification can be accomplished using many techniques. Surface modification by compounds of silicon, phosphorous, boron, antimony, coke, and magnesium are documented. However, while molecular sieve surface modification techniques have been used to successfully deactivate the external surface of a molecular sieve or to improve selectivity of a molecular sieve, molecular sieve surface modification techniques typically cannot attain both an enhanced overall activity and an improved selectivity. Specifically, such techniques for increasing or decreasing external surface activity, as desired, typically detrimentally affect internal surface activity. As a result, chemical processing using the surface-modified molecular sieve may be impaired by either reduced overall catalytic activity or a loss in selectivity due to the surface modification.

Such molecular sieve surface modification techniques have not resulted in molecular sieves having both deactivated or passivated external active sites for a decreased external surface activity and pores that can accurately differentiate molecules to facilitate catalysis applications and adsorptive processes of interest, such as para-alkyl selectivation. As used herein, the term “para-alkyl selectivation” refers to modifying a catalyst or catalytic reaction system so that it preferentially forms more para-substituted dialkylbenzenes than the expected equilibrium proportions relative to the other isomers.

Accordingly, it is desirable to provide surface-modified molecular sieve catalysts and methods for preparing the same. In addition, it is desirable to provide surface-modified molecular sieves that have such desired surface properties as a decreased external surface activity while maintaining or enhancing internal surface activity, which properties are useful in certain catalytic applications and adsorptive processes, such as para-alkyl selectivation. Furthermore, other desirable features and characteristics will become apparent from the subsequent detailed description and the appended claims, taken in conjunction with the accompanying drawings and the foregoing technical field and background.

### BRIEF SUMMARY

Bismuth-modified molecular sieve catalysts and methods for preparing and using bismuth-modified molecular sieve catalysts are provided. In accordance with one exemplary embodiment, a bismuth-modified molecular sieve catalyst includes a molecular sieve having an external surface. The molecular sieve is selected from the group consisting of MFI, MEL, MOR, MTW, BEA, CHA, FAU, EMT, MTT, MWW, TON, TUN, EUO, IMF and FER framework types. The bismuth-modified molecular sieve catalyst further includes a coating on the external surface. The coating comprises a bismuth-containing material.

In another embodiment, a method for preparing a bismuth-modified molecular sieve catalyst is provided. The method for preparing a bismuth-modified molecular sieve catalyst includes depositing a bismuth-containing material on an external surface of a molecular sieve. The molecular sieve is selected from the group consisting of MFI, MEL, MOR, MTW, BEA, CHA, FAU, EMT, MTT, MWW, TON, TUN, EUO, IMF and FER framework types. The method further includes dispersing the bismuth-containing material on the external surface of the molecular sieve to deactivate the external surface.

In accordance with another exemplary embodiment, a method for using a bismuth-modified molecular sieve catalyst is provided. The method for using a bismuth-modified molecular sieve catalyst includes providing an MFI zeolite catalyst having an external surface selectively modified with bismuth and having an internal surface. The method contacts an aromatic and a reactant over the MFI zeolite catalyst to produce product compounds. The surface activity of the MFI zeolite is greater at the internal surface than at the external surface. Pore sizes of the MFI zeolite limit diffusion of a non-selected product compound relative to diffusion of a selected product compound to facilitate selective recovery of a product with a selected product compound content of greater than about 80 weight percent (wt %).

### BRIEF DESCRIPTION OF THE DRAWINGS

Embodiments of bismuth-modified molecular sieve catalysts and methods for their preparation and use will hereinafter be described in conjunction with the following drawing figures wherein:

FIGS. 1 and 2 are flow diagrams illustrating methods for preparing a bismuth-modified molecular sieve catalyst according to exemplary embodiments.

### DETAILED DESCRIPTION

The following detailed description is merely exemplary in nature and is not intended to limit the bismuth-modified molecular sieve catalyst or the methods of preparing or using the bismuth-modified molecular sieve catalyst claimed herein. Furthermore, there is no intention to be bound by any theory presented in the preceding background or the following detailed description.

As described herein, an external surface of a molecular sieve is modified with bismuth to decrease surface activity, i.e., the external surface is deactivated. The external surfaces of molecular sieves herein are deactivated by forming a bismuth compound thereon. As used herein “deactivate” refers both to decreasing activity and to eliminating activity, i.e., “deactivate” covers both partial and complete deactivation. In exemplary embodiments, an internal surface of the molecular sieve is not deactivated by the bismuth compound. As used conventionally, an “external surface” of a molecular sieve is formed at the perimeter of the molecular sieve, and an “internal surface” of a molecular sieve bounds a pore within the molecular sieve. The external surface and internal surface may be considered to meet at each pore opening. Deactivation of the internal surface is prevented herein because the bismuth compound is too large to enter the pores in the molecular sieves. As a result, the bismuth-modified molecular sieve catalyst has the same largest cavity diameter and maximum limiting pore diameter as the non-modified molecular sieve before modification with bismuth. Therefore, the surface modification with bismuth does not reduce the activity and inherent selectivity of the molecular sieve.

An exemplary embodiment of the bismuth-modified molecular sieve catalyst is formed from an MFI zeolite and a coating of bismuth (III) oxide (Bi₂O₃). The bismuth-modified molecular sieve catalyst may comprise a formed molecular sieve, as hereinafter described. A bismuth-modified molecular sieve catalyst formed from an MFI zeolite and a bismuth oxide coating may be used to catalyze a reaction of an aromatic to product compounds, and to selectively recover selected product compounds. Specifically, the bismuth-modified molecular sieve catalyst has a deactivated external surface that does not catalyze the aromatic reaction. The internal surfaces do catalyze the aromatic reaction and form the selected and non-selected product compounds at equilibrium conditions. However, the pores limit diffusion of larger product compounds relative to diffusion of smaller product compounds. Therefore, the product recovered from the reaction before the larger product compounds diffuse from the pores includes a greater than equilibrium amount of smaller product compounds. For example, the recovered product may have a smaller product content of greater than 80 wt %. Molecules of para-xylene are smaller than molecules of meta-xylene and ortho-xylene. Therefore, the bismuth-modified molecular sieve catalyst may be effective to prepare a xylene product having a para-xylene content of greater than 80 wt %.

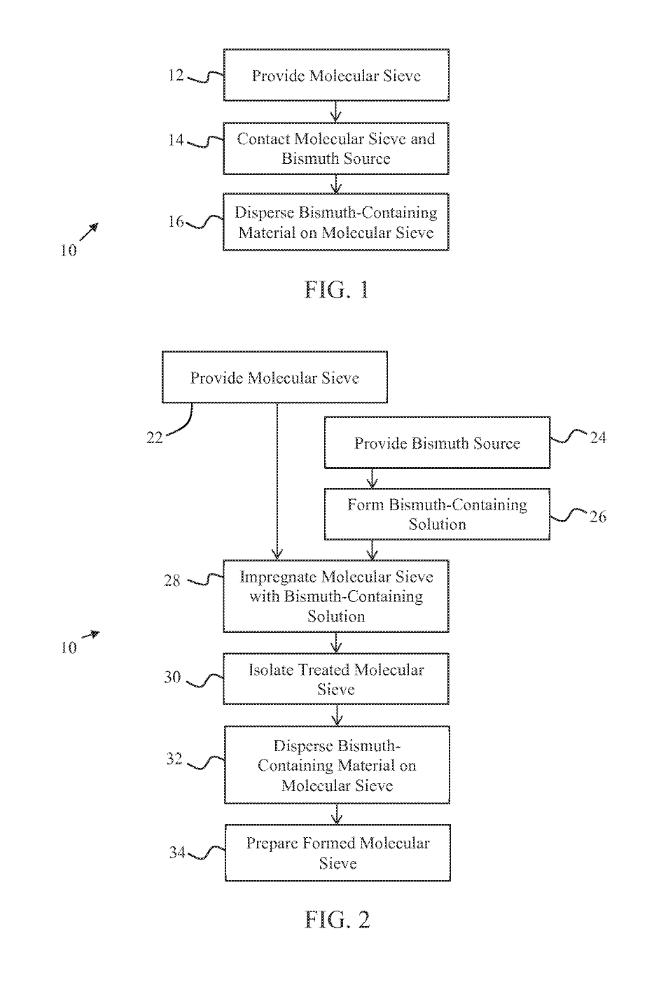

FIG. 1 is a flow diagram of a method 10 for preparing a bismuth-modified molecular sieve catalyst in accordance with an exemplary embodiment. The exemplary method 10 begins by providing a molecular sieve at step 12. The molecular sieve may have any suitable crystalline framework, crystal size, pore size, and composition as is suitable for its catalytic use. In an exemplary embodiment, the molecular sieve is a zeolite having a silica:alumina ratio of from about 2 to about 500, such as about 2 to about 200. An exemplary molecular sieve has an average particle diameter of from about 0.1 to about 200 microns, such as about 1 micron to about 100 microns. As used herein, the average particle diameter is the diameter of a circle having the same area as the average cross sectional area of a particle. An exemplary molecular sieve has a largest cavity diameter of no more than about 10.0 Angstrom (Å), such as from about 5.0 Å to about 10.0 Å, such as from about 6.0 Å to about 8.0 Å, for example about 7.0 Å. As used herein, the “largest cavity diameter” is the diameter of a circle having the same area as the largest section of a given pore. An exemplary molecular sieve has a maximum limiting pore diameter no more than about 8.0 Å, such as from about 2.0 Å to about 8.0 Å, such as from about 4.0 Å to about 6.0 Å, for example about 5.0 Å. As used herein, the limiting diameter is the diameter of a circle having the same area as the smallest section of a given pore.

According to exemplary embodiments, the molecular sieve comprises a crystalline alumino-silicate zeolite having a porous oxide structure with a well-defined pore structure due to the high degree of crystallinity. Suitable exemplary zeolites include those having structure type MFI (e.g., ZSM-5; U.S. Pat. No. 3,702,886), MEL (e.g., ZSM-11; U.S. Pat. No. 3,709,979), MTW (e.g., ZSM-12; U.S. Pat. No. 3,832,449), TON (e.g., ZSM-22), MTT (e.g., ZSM-23; U.S. Pat. No. 4,076,842), FER (e.g., ZSM-35; U.S. Pat. No. 4,016,245), EUO (e.g., ZSM-50), FAU (e.g., Zeolites X; U.S. Pat. No. 2,882,244 and Y; U.S. Pat. No. 3,130,007), EMT, IMF, TUN, MEI, MSE, and BEA (Beta). Additionally, suitable exemplary zeolites include UZM zeolites available from UOP LLC (Des Plaines, Ill. (USA)) and covered under one or more of the following U.S. patents or published applications: U.S. Pat. Nos. 6,419,895; 6,613,302; 6,776,975; 6,713,041; 6,756,030; 7,344,694; 6,752,980; 6,982,074; 6,890,511; 7,575,737; US 20080170987; and US 20080031810. An exemplary zeolite comprises an MFI zeolite available from UOP, LLC. Zeolite structure types are described in “Atlas of Zeolite Structure Types”, W. M. Meier, D. H. Olson and C. Baerlocher, 5th revised edition, 2001, Elsevier. The zeolites have ion exchangeable sites on the internal and external surfaces of the zeolite. Sodium occupies ion exchangeable sites within and on the surface of sodium-form zeolites, ammonium (NH₄) occupies ion exchangeable sites within and on the surface of ammonium-form zeolites (or “NH₄-zeolite”), and hydrogen occupies the ion exchangeable sites within and on the surface of hydrogen-form zeolites, each in the range of about 0.1 wt % to about 20 wt %, by weight of the zeolite.

In one embodiment, the zeolite (as powder or as a “formed zeolite”) may be commercially available and thus obtained by commercial sources. Alternatively, the zeolite may be synthesized according to known methods, such as by crystallizing a silica-alumina gel composition formed from an alumina source such as sodium aluminate, Boehmite, aluminum alkoxides such as aluminum-isopropyloxide, aluminum sec-butoxide, aluminum trihydroxide, or the like and a silica source such as sodium silicate, alkyl silicates such as tetraethyl orthosilicate and the like and commercially available silica sources. Other alumino-silicates such as kaolin are used as well. The alumina and silica may be dissolved with a template as known in the art in a basic environment, such as, for example, a sodium hydroxide aqueous solution, and crystallized at from about 70° C. to about 300° C., such as from about 75° C. to about 200° C., for example at about 100° C. After crystallization, the zeolite is in a sodium form. Zeolite synthesis using a template to direct the formation of specific zeolite topologic framework structures is known to those skilled in the art. Some common structure directing agents (templates) include organo ammonium cations selected from quaternary ammonium cations, protonated amines, diquaternary ammonium, and the like. The use of structure directing agents, however, is optional in synthesis of some zeolites (e.g., MFI, zeolite X).

The sodium form of the zeolite may optionally be ion exchanged with ammonium to form a “NH₄-form zeolite”. In this regard, the sodium-form zeolite is exposed to an ammonium-comprising solution, such as, for example, a NH₄NO₃ solution, for ion-exchange to produce the NH₄-form zeolite. In an exemplary embodiment, substantially all of the ion-exchangeable Na sites of the zeolite are exchanged with NH₄ such that the weight percent of Na in the NH₄-form of the zeolite is less than about 0.5% (on a volatile free basis).

Once formed, the zeolite may be dried by known drying methods. For example, the zeolite may be dried overnight, such as in flowing nitrogen at 200° C. and cooled in dry nitrogen to 50° C. Alternatively, the zeolite may be calcinated. Calcination of a NH₄-form zeolite results in a hydrogen-form zeolite. Calcination may be performed at temperatures of from about 400° C. to about 600° C., such as at about 550° C., under inert atmosphere and/or air using a heating rate of about 0.5° C./minute to about 10° C./minute, such as at about 2° C./minute for about one to about ten hours, such as for about four hours.

In an exemplary embodiment, the zeolite is a zeolite powder. In other embodiments, the zeolite may be a “formed zeolite”. A “formed zeolite” comprises the zeolite powder bound with an inert binder. In an exemplary embodiment of a formed zeolite, the inert binder may include those well known in the art such as silica, alumina, titania, zirconia alumino-phosphate (ALPO) binder, and combinations thereof. For example, the formed zeolite may be prepared into extrudates by forming methods well known in the art. The extrudates may be comprised of from about 35 to about 90 wt % of powdered zeolite and about 10 to about 65 wt % of inert binder (on a volatile-free basis). An exemplary binder concentration comprises about 12 to about 30 wt % of the zeolite. Rather than formed zeolites, the zeolite powder may be formed with the inert binder into beads, tablets, macrospheres, extrudates, oil drops, microspheres, spheres, beads or the like. Such forming may occur prior to or after ion-exchange, as previously described. Further, the zeolite powder may be prepared as a formed zeolite after surface modification with bismuth as described below. While processing above has been described in relation to zeolites, the method may include similar processing suitable for other molecular sieves.

The exemplary method may continue with contacting the molecular sieve with a bismuth source at step 14. Exemplary bismuth sources include bismuth oxides, bismuth nitrates, and other bismuth salts. During contact, a bismuth-containing material is formed on the external surface of the molecular sieve to form a treated molecular sieve. In an exemplary embodiment, the bismuth-containing material is a bismuth oxide, such as bismuth (III) oxide (Bi₂O₃). The exemplary bismuth-containing material is too large to enter the pores of the molecular sieve; therefore, the internal surface of the molecular sieve is essentially free of the bismuth-containing material, i.e., the molecular sieve retains at least about 75% of pore volume and at least about 75% of internal surface area relative to the molecular sieve before modification. In certain embodiments, the molecular sieve retains from about 75% to about 100%, such as more than about 85% or more than 90%, of pore volume and from about 75% to about 100%, such as more than about 85% or more than 90%, of internal surface area. In an exemplary embodiment, the molecular sieve retains substantially 100% of pore volume and substantially 100% of internal surface area.

The exemplary method may continue in FIG. 1 with dispersing the bismuth-containing material on the external surface of the treated molecular sieve at step 16. For example, the molecular sieve may be heated to cause the bismuth-containing material to soften and form a thin layer on the external surface of the treated molecular sieve. The dispersed layer of the bismuth-containing material covers active sites on the external surface of the molecular sieve, effectively deactivating, partially or completely, those external active sites. Because the bismuth-containing material is not present in the pores, active sites on the internal surface of the molecular sieve are not deactivated, but remain active. In an exemplary embodiment, the bismuth-containing material is bismuth (III) oxide, which has a melting point of about 820° C. For such an embodiment, the molecular sieve may be heated to over 600° C., such as to about 800° C. The exemplary process may heat the molecular sieve for about one hour to about four hours, such as for two hours. Such moderate heating typically does not cause any crystallinity loss in the molecular sieve.

FIG. 2 illustrates a method 10 for preparing a bismuth-modified molecular sieve catalyst in accordance with another exemplary embodiment. The exemplary method 10 again begins by providing a molecular sieve at step 22 as described in relation to step 12 above. Further, FIG. 2 illustrates processing of a bismuth source. Specifically, at step 24 a bismuth source is provided. An exemplary bismuth source may be a bismuth oxide, a bismuth nitrate or sub-nitrate, or other bismuth salts, such as bismuth (III) sulfate, or bismuth chloride (III). Other bismuth sources may be suitable if the sources provide for the formation of a bismuth-containing material on a molecular sieve external surface as described below. An exemplary bismuth source is bismuth (III) nitrate (Bi(NO₃)₃).

In the exemplary embodiment, a bismuth-containing solution is formed at step 26. Specifically, the bismuth source is dissolved by a solvent to form a bismuth-containing solution. An exemplary solvent is a mineral acid solution, such as nitric acid, hydrochloric acid, sulfuric acid, or perchloric acid. An exemplary solvent is provided as an aqueous solution. For example, the solvent may be a 1 molar solution of the acid. In an exemplary embodiment, the solvent is a 1 M nitric acid solution.

Step 28 impregnates the molecular sieve with the bismuth-containing solution to form a mixture. Upon contact between the molecular sieve and the solution, a bismuth-containing material is formed through precipitation or removal of solvent by evaporation. Bismuth components of the solution are too large to enter the pores and do not contact the internal active sites of the molecular sieve. Therefore, precipitation of the bismuth-containing material causes formation of the bismuth-containing material on the external surfaces of the molecular sieve to form a treated molecular sieve. For example, the bismuth-containing material may be deposited on or adsorbed by the external surface of the molecular sieve.

Precipitation may be caused by changes in the solution after contact with the molecular sieve. For example, the solution may undergo a change in composition such as a change in water content, or a change in pH. In an exemplary embodiment, a 1 M nitric acid solution with dissolved bismuth (III) nitrate undergoes a change in pH upon contact with the active sites of the molecular sieve. Specifically, the pH may change from a pH of about 0 to a pH of from about 3 to about 8. With the change in pH, a bismuth-containing material precipitates out of solution and is deposited on the external surface of the molecular sieve. For example, a bismuth oxide forms on the external surface of the molecular sieve. In exemplary embodiments, bismuth (III) oxide forms on the external surface of the molecular sieve.

Contact between the molecular sieve and the bismuth-containing solution may occur for from about 1 hour to about 4 hours. Further, the mixture of the molecular sieve and the bismuth-containing solution may be maintained at a temperature of about 20° C. to about 90° C. Thereafter, the treated molecular sieve is isolated at step 30. Specifically, the treated molecular sieve may be removed from the remaining solution or supernatant. In an exemplary embodiment, the treated molecular sieve is recovered from the supernatant by removing the liquid phase by known liquid/solid separation techniques such as filtration, distillation, solvent evaporation, or the like. The separated treated molecular sieve is then optionally dried. Drying may be performed at temperatures of from about 60° C. to about 200° C. The drying time ranges from about one hour to about 24 hours.

After the bismuth-containing material is isolated, step 32 disperses the bismuth-containing material on the external surfaces of the molecular sieve. As described above in relation to step 16, the molecular sieve may be heated to cause the bismuth-containing material to soften and form a thin layer on the external surface of the treated molecular sieve. The dispersed layer of the bismuth-containing material covers active sites on the external surface of the molecular sieve, effectively deactivating, partially or completely, those external active sites. Because the bismuth-containing material is not present in the pores, active sites on the internal surface of the molecular sieve are not deactivated, but remain active. In an exemplary embodiment, the bismuth-containing material is bismuth (III) oxide, which has a melting point of about 820° C. For such an embodiment, the molecular sieve may be heated to about 800° C. for about one hour to about four hours, such as for two hours. Such moderate heating typically does not cause any crystallinity loss in the molecular sieve.

After dispersion of the bismuth-containing material on the external surfaces of the treated molecular sieve, the treated molecular sieve may optionally be ion exchanged with ammonium to form a NH₄-form treated molecular sieve. In this regard, the treated molecular sieve is exposed to an ammonium-comprising solution, such as, for example, a NH₄NO₃ solution, for ion-exchange to produce a NH₄-form treated molecular sieve. In an exemplary embodiment, substantially all of the ion-exchangeable Na sites of the internal surfaces of the treated molecular sieve are exchanged with NH₄.

When the molecular sieve is provided in step 22 as a powder, the exemplary method 10 may continue with formation of a “formed treated molecular sieve” at step 34. A “formed treated molecular sieve” comprises the treated molecular sieve powder bound with an inert binder. In an exemplary embodiment of a formed treated molecular sieve, the inert binder may include those well known in the art such as silica, alumina, titania, zirconia, alumino-phosphate (ALPO) binder, and combinations thereof. For example, the formed treated molecular sieve may be prepared into extrudates by forming methods well known in the art. The extrudates may be comprised of from about 35 to about 90 wt % of powdered treated molecular sieve and about 10 to about 65 wt % of inert binder (on a volatile-free basis). An exemplary binder concentration comprises about 12 to about 30 wt % of the bismuth-modified molecular sieve catalyst. Rather than formed molecular sieves, the molecular sieve powder may be formed with the inert binder into beads, tablets, macrospheres, extrudates, oil drops, microspheres, spheres, beads or the like. Such forming may occur prior to or after the optional ion-exchange of the treated molecular sieve.

Bismuth-modified molecular sieve catalysts formed according the exemplary methods described in relation to FIGS. 1 and 2 can exhibit increased selectivity for desired species during selected reactions while exhibiting little or no loss in activity as compared to the molecular sieve before surface modification. Specifically, non-selective active sites on the external surfaces of the molecular sieve are deactivated without inhibiting the selective active sites on the internal surfaces of the molecular sieve, either through directly affecting the selective active sites or by rendering the selective active sites inaccessible by blocking pore openings. In certain embodiments, the non-selective active sites have reduced activity relative to the selective active sites, i.e., internal active sites have greater activity than external active sites. Also, in certain embodiments, the bismuth-modified molecular sieve catalyst retains the same largest cavity diameter and the same maximum limiting pore diameter as the molecule sieve before surface modification.

Exemplary bismuth-modified molecular sieve catalysts may be particularly effective for processing aromatics. For example, such catalysts may be useful for catalyzing toluene disproportionation reactions, toluene methylation reactions, ethylbenzene dealkylation reactions, and other relevant aromatic reactions. During an exemplary toluene disproportionation reaction, the bismuth-containing material blocks contact between toluene and the external surface of the molecular sieve. The toluene enters the pores and upon contact with the active sites on the internal surfaces of the molecular sieves, a reaction is catalyzed and converts the toluene to benzene, para-xylene, meta-xylene and ortho-xylene at equilibrium. Due to the smaller sizes of the benzene and para-xylene molecules relative to the meta-xylene and ortho-xylene molecules, benzene and para-xylene diffuse out of the pores more readily than the meta-xylene and ortho-xylene. As a result, a product stream of benzene and para-xylene may be recovered and separated into a benzene product and a xylene product. In exemplary embodiments, the xylene product includes more than about 80 wt % para-xylene, such as more than about 85 wt % para-xylene. For example, the xylene product may include from about 80 wt % to about 99 wt %, such as from about 85 wt % to about 90 wt %.

In an exemplary embodiment, a method for using the bismuth-modified molecular sieve catalyst includes locating in a reaction zone a molecular sieve having external surfaces selectively modified with bismuth and internal surfaces that are not modified with bismuth. A stream of aromatics and a reactant, such as hydrogen, is flowed into contact with the catalyst. As the aromatics and the reactant contact over the catalyst, a reaction is catalyzed at the internal surfaces and forms xylenes. The reaction at the internal surfaces results in an equilibrium amount of xylene isomers. However, the pore openings limit diffusion of meta-xylene and ortho-xylene from the pores. Para-xylene diffuses more quickly from the pores. The method includes recover of a xylene product with a para-xylene content of greater than about 80 wt %.

### Examples

The following are examples of bismuth-modified molecular sieve catalysts, in accordance with exemplary embodiments described herein. The examples are provided for illustration purposes only, and are not meant to limit the various embodiments herein in any way.

For Sample 1, five grams of MFI zeolite in powder form was provided. The MFI zeolite was large crystal, e.g., it had an average particle diameter greater than about 1 micron. Further, the MFI zeolite had a Si:Al ratio of 40:1. A bismuth source was provided in the form of a bismuth-containing solution. Specifically, the bismuth-containing solution was formed by dissolving 10 grams of Bi(NO₃)₃.5H₂O in 50 grams of 1 M nitric acid. The zeolite and bismuth source were contacted by impregnating the zeolite in 7.5 grams of the bismuth-containing solution to form a mixture. The zeolite and bismuth source were contacted for about 120 minutes. During contact, bismuth (III) oxide was deposited on the external surfaces of the zeolite to form a treated zeolite. Then, the mixture was dried at room temperature overnight. The treated zeolite was calcinated at 800° C. for 2 hours, underwent ammonium ion exchange, and was bound with a colloidal silica binder.

Sample 2 includes the same MFI zeolite modified with lanthanum and magnesium rather than bismuth and bound with colloidal silica according to conventional processes. Sample 3 includes the same MFI zeolite without any surface-modification. Table I compares selectivity and activity for Samples 1-3 when catalyzing a toluene disproportionation reaction to form benzene and xylene isomers at five different reaction temperatures. “Selectivity” refers to the percentage of para-xylene in the total recovered xylene content. “Activity” refers to the percentage of toluene converted to other products, including benzene and xylenes.

TABLE I

| | | Reaction Temp: | | | | |
|---|---|---|---|---|---|---|
| | | 400° C. | 425° C. | 450° C. | 475° C. | 500° C. |
| Sample 1: | Selectivity | 79% | 81% | 84% | 85% | 85% |
| Bismuth- | Activity | 1.6% | 2.6% | 3.9% | 5.2% | 7.0% |
| modified | | | | | | |
| zeolite | | | | | | |
| Sample 2: | Selectivity | 0.0% | 100% | 100% | 78% | 79% |
| La and Mg | Activity | 0.2% | 0.5% | 0.8% | 1.5% | 2.6% |
| modified | | | | | | |
| zeolite | | | | | | |
| Sample 3: | Selectivity | 49% | 48% | 48% | 42% | 42% |
| Non- | Activity | 0.9% | 1.1% | 1.7% | 2.7% | 4.2% |
| modified | | | | | | |
| zeolite | | | | | | |

As shown in Table I, the bismuth-modified zeolite exhibited a much higher toluene conversion rate at all reaction temperatures, and a selectivity preference for the formation of para-xylene over other xylenes twice that of the non-modified zeolite at higher reaction temperatures. The lanthanum and magnesium modified zeolite exhibited high selectivity for para-xylene, but at a much lower toluene conversion rate than the bismuth-modified zeolite.

In a second example, Samples 4 was left unmodified while Samples 5 and 6 were prepared similarly to Sample 1. Specifically, Sample 4 was provided from an MFI zeolite commercially available from Tosoh Corp. of Japan. The MFI zeolite was in powder form with a Si:Al ratio of 19:1. Sample 5 was prepared from the same MFI zeolite and was modified to form a treated zeolite comprised of 1% Bi₂O₃. After bismuth dispersion, the treated zeolite was bound with alumina binder. Sample 6 was prepared from the same MFI zeolite and was bound with alumina before being modified to form a treated zeolite comprised of 2% Bi₂O₃.

Table II describes selectivity and activity for Samples 4-6 when catalyzing an ethylbenzene dealkylation reaction from a feed of benzene and xylenes to form benzene and C₂ at four different reaction temperatures. In this process, the feed contains at least about 60% meta-xylene, at least about 20% ortho-xylene, and about 5% ethylbenzene. The objective of the reaction process is to dealkylate ethylbenzene (on internal acid sites) without losing xylenes through xylene disproportionation on external surface acid sites. “Selectivity” refers to the percentage of xylene loss. Xylene loss is determined as 100%*[Xylene(initial)-xylene(final)]/xylene(initial). Low xylene loss means higher reaction selectivity. “Activity” refers to the percentage of ethylbenzene that is dealkylated. Activity is determined as 100%*[Ethylbenzene(initial)-Ethylbenzene(final)]/ethylbenzene(initial).

TABLE II

| | | Reaction Temp: | | | |
|---|---|---|---|---|---|
| | | 260° C. | 280° C. | 300° C. | 320° C. |
| Sample 4: Non- | Xylene loss | | 0.13% | 0.29% | 0.60% |
| modified zeolite | Activity | | 2.7% | 4.8% | 7.6% |
| Sample 5: Zeolite | Xylene loss | 0.07% | 0.13% | 0.27% | |
| with 1% Bi₂O₃ | Activity | 3.3% | 4.0% | 6.0% | |
| modified before | | | | | |
| binder | | | | | |
| Sample 6: Zeolite | Xylene loss | | 0.04% | 0.15% | 0.36% |
| with 2% Bi₂O₃ | Activity | | 3.0% | 6.4% | 11.2% |
| modified after | | | | | |
| binder | | | | | |

As shown in Table II, the bismuth-modified zeolites exhibited greater selectivity as the loss by the non-modified zeolite was greater than Sample 5 at each conversion activity, and ranged from 66% greater to 325% greater than Sample 6 at each reaction temperature. Further, activity exhibited by bismuth-modified zeolites was higher than activity of the non-modified zeolite across all reaction temperatures. In view of the dramatic reduction in xylene loss from the non-modified zeolite, the bismuth-modified zeolites exhibit improved performance for ethylbenzene dealkylation.

As described herein, bismuth-modified molecular sieve catalysts and methods for preparing and using bismuth-modified molecular sieve catalysts have been provided. The bismuth-modified molecular sieve catalysts and methods for preparing and using bismuth-modified molecular sieve catalysts provide for deactivating non-selective sites at external surfaces of molecular sieves. Selective active sites at internal surfaces of molecular sieves are not deactivated by the bismuth deposition and dispersion process. As a result, the molecular sieve catalyst may exhibit improved selectivity while minimizing or avoiding activity loss. Selectivity provided by the limited openings of the pores is not impaired by the exemplary bismuth surface-modification.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the claimed subject matter in any way. Rather, the foregoing detailed description will provide those skilled in the art with a convenient road map for implementing an exemplary embodiment or embodiments. It being understood that various changes may be made in the function and arrangement of elements described in an exemplary embodiment without departing from the scope set forth in the appended claims.

What is claimed is:

1. A bismuth-modified molecular sieve catalyst comprising:
a molecular sieve having an external surface, wherein the molecular sieve is selected from the group consisting of MFI, MEL, MOR, MTW, BEA, CHA, FAU, EMT, MTT, MWW, TON, TUN, EUO, IMF and FER framework types; and
a coating on the external surface, wherein the coating comprises a bismuth-containing material.

2. The bismuth-modified molecular sieve catalyst of claim 1 wherein the coating comprises bismuth oxide.

3. The bismuth-modified molecular sieve catalyst of claim 1 wherein the coating consists essentially of bismuth oxide.

4. The bismuth-modified molecular sieve catalyst of claim 1 wherein the coating consists of bismuth oxide.

5. The bismuth-modified molecular sieve catalyst of claim 1 wherein the bismuth-modified molecular sieve catalyst is bound to a binder.

6. The bismuth-modified molecular sieve catalyst of claim 1 wherein the molecular sieve is an MFI zeolite.

7. The bismuth-modified molecular sieve catalyst of claim 6 wherein the MFI zeolite has an average particle diameter of from about 0.1 micron to about 100 microns.

8. The bismuth-modified molecular sieve catalyst of claim 7 wherein the MFI zeolite has a largest cavity diameter of no more than about 10.0 Å and a maximum limiting pore diameter of no more than about 8.0 Å.

9. The bismuth-modified molecular sieve catalyst of claim 8 wherein the bismuth-modified molecular sieve catalyst has a modified largest cavity diameter equal to the largest cavity diameter and a modified maximum limiting pore diameter equal to the maximum limiting pore diameter.

10. A method for preparing a bismuth-modified molecular sieve catalyst, the method comprising the steps of:
depositing a bismuth-containing material on an external surface of a molecular sieve selected from the group consisting of MFI, MEL, MOR, MTW, BEA, CHA, FAU, EMT, MTT, MWW, TON, TUN, EUO, IMF and FER framework types; and
dispersing the bismuth-containing material on the external surface of the molecular sieve to deactivate the external surface.

11. The method of claim 10 wherein the molecular sieve is a powder and wherein depositing a bismuth-containing material on the external surface of the molecular sieve comprises:
forming a bismuth-containing aqueous solution from a bismuth source and a solvent;
mixing the molecular sieve with the bismuth-containing aqueous solution to form a mixture; and
removing the solvent from the mixture.

12. The method of claim 11 wherein mixing the molecular sieve with the bismuth-containing aqueous solution to form the mixture comprises contacting the molecular sieve and the bismuth-containing aqueous solution under conditions sufficient to deposit the bismuth-containing material on the external surface of the molecular sieve to form a treated molecular sieve.

13. The method of claim 12 wherein removing the solvent from the mixture comprises drying the treated molecular sieve, and wherein the method further comprises binding the treated molecular sieve with colloidal silica.

14. The method of claim 12 further comprising performing an ammonium exchange with the treated molecular sieve.

15. The method of claim 11 wherein dispersing the bismuth-containing material on the external surface of the molecular sieve comprises heating the mixture to a temperature of greater than about 600° C. for a duration of greater than about 1 hour.

16. The method of claim 11 wherein forming the bismuth-containing aqueous solution comprises forming the bismuth-containing aqueous solution from Bi(NO₃)₃ and a solvent, and wherein mixing the molecular sieve with the bismuth-containing aqueous solution to form the mixture comprises precipitating bismuth oxide onto the external surface of the molecular sieve.

17. The method of claim 11 wherein the molecular sieve has a maximum pore size, wherein forming the bismuth-containing aqueous solution comprises forming an aqueous solution of Bi(NO₃)₃, wherein mixing the molecular sieve with the bismuth-containing aqueous solution to form the mixture comprises precipitating a bismuth oxide from the bismuth-containing aqueous solution onto the molecular sieve, and wherein the bismuth oxide has a minimum dimension larger than the maximum pore size.

18. The method of claim 10 wherein depositing the bismuth-containing material on the external surface of the molecular sieve comprises depositing a bismuth oxide on the external surface of an MFI zeolite having an average particle diameter of from about 0.1 micron to about 100 microns.

19. A method for using a bismuth-modified molecular sieve catalyst, the method comprising the steps of:
providing an MFI zeolite catalyst having an external surface selectively modified with bismuth and having an internal surface; and
contacting an aromatic and a reactant over the MFI zeolite catalyst to produce product compounds, wherein surface activity of the MFI zeolite catalyst is greater at the internal surface than at the external surface, and wherein pore sizes of the MFI zeolite catalyst limit diffusion of a non-selected product compound relative to diffusion of a selected product compound to facilitate selective recovery of a product with a selected product compound content of greater than about 80 wt %.

20. The method of claim 19 wherein contacting the aromatic and the reactant over the MFI zeolite catalyst to produce product compounds comprises producing xylene isomers, and wherein the selected product compound is para-xylene.
